(19) United States
(12) Patent Application Publication
(10) Pub. No.: US 2010/0174196 A1
(43) Pub. Date: Jul. 8, 2010

(54) SYSTEMS AND METHODS FOR GUIDING THE ANALYSIS AND TREATMENT OF A BODY LUMEN

(75) Inventors: S. Eric Ryan, Hopkinton, MA (US); Jing Tang, Arlington, MA (US)

Correspondence Address: MILLS & ONELLO LLP, ELEVEN BEACON STREET, SUITE 605, BOSTON, MA, 02108, US

(73) Assignee: CORNOVA, INC., Burlington, MA (US)

(21) Appl. No.: 12/663,166

(22) PCT Filed: Jun. 20, 2008
(86) PCT No.: PCT/US08/67669

§ 371 Date: Dec. 4, 2009

Related U.S. Application Data
(60) Provisional application No. 60/945,481, filed on Jun. 21, 2007.
(60) Provisional application No. 61/019,626, filed on Jan. 8, 2008.
(60) Provisional application No. 61/025,514, filed on Feb. 1, 2008.

(51) Int. Cl. A61B 6/00; A61N 5/06; A61M 29/00; A61F 2/84

(52) U.S. Cl. 600/473; 607/88; 604/96.01; 623/1.11

(57) ABSTRACT

A system for treating a body lumen comprises a catheter including a flexible conduit that is elongated along a longitudinal axis and suitable for insertion into a body lumen, the conduit having a proximal end and a distal end, one or more waveguides integrated with the flexible conduit, the one or more waveguides constructed and arranged to deliver and collect radiation concentrated along a predetermined radial axis of the conduit, the predetermined radial axis of the conduit substantially aligned with respect to at least one therapy delivery component of the catheter, at least one radiation source connected to a transmission input of the one or more waveguides integrated with the flexible conduit, and at least one optical detector connected to a transmission output of the one or more waveguides integrated with the flexible conduit.

### RELATED APPLICATIONS

This application claims the benefit of U.S. Patent Application No. 60/945,481, filed on Jun. 21, 2007, U.S. Patent Application No. 61/019,626, filed on Jan. 8, 2008, and U.S. Patent Application No. 61/025,514, filed on Feb. 1, 2008, the contents of each of which is incorporated herein by reference in its entirety. This application is related to U.S. patent application Ser. No. 11/537,258, filed on Sep. 29, 2006, published as Patent Application Publication No. 2007/0078500 A1, and U.S. patent application Ser. No. 11/834,096, filed on Aug. 6, 2007, the entire contents of each of which is herein incorporated by reference.

### BACKGROUND OF THE INVENTION

1. Field of the Invention

Embodiments of the present invention are directed to systems and methods for the treatment of body lumens. More particularly, the present invention relates to catheter systems for treatment and/or diagnosis of vessels, including those relating to angioplasty treatment.

2. Description of the Related Art

Stents are implantable prosthesis used to maintain and/or reinforce vascular and endoluminal ducts in order to treat and/or prevent a variety of medical conditions. Typical uses include maintaining and supporting coronary arteries after they are opened and unclogged, such as through an angioplasty operation. A stent is typically deployed in an unexpanded or crimped state using a catheter and, after being properly positioned within a vessel, is then expanded into its final shape (such as with an expandable balloon incorporated into the catheter).

As a foreign object inserted into a vessel, a stent can potentially impede the flow of blood. This effect can cause or exacerbate undesired growth of tissue on and around the stent, potentially leading to complications including thrombosis and restenosis. The likelihood of such problems is significantly increased as a result of a stent's non-conformity with a vessel's walls when expanded. Thus, stent systems are generally designed to minimize the impedance of a vessel by including a minimal level of strut material, by being flexible in order to conform to a vessel's walls. Typical materials for stent struts include stainless steel, cobalt-chromium, and nitinol.

Many stenting procedures are further challenged when targeting occlusions around vessel bifurcations or other highly curved tracts. Some methods attempt to bend stents to conform to the tract. Such a procedure can be difficult with a traditional balloon catheter because a fully expanded balloon will typically form a straight, highly inflexible tubular body that will resist compliance to the vessel's natural shape. This can lead to a stent being non-conformant with the vessel or bifurcation area and can cause undesired damage to the vessel's walls and further impede blood flow.

Rather than attempt to bend a single stent to conform to a tenuously curved area, multiple overlapping stents have been placed about the area in order to avoid some of the above described challenges. However, the use of multiple stents, e.g. in a “kissing stent” bifurcation, can lengthen and complicate a procedure, adding additional risk and expense. The overlapping portions of the stents may also unnecessarily add obstructive stent material, potentially interfering with blood flow and increasing the likelihood of complications such as restonosis.

Some stent bifurcation systems are designed with stents having “trap doors” or other openings in order to avoid blocking vessel branch openings or for allowing passage of subsequent stents. Such systems are proposed in, for example, US patent publication No. 2004/0176837 A1, incorporated herein by reference in its entirety. These systems, however, can typically require expensive and/or complex deployment components or procedures. Because positioning of these systems generally requires an accurate rotational component and because traditional positioning methods (e.g. fluoroscopy) generally do not provide for accurate rotational placement within a vessel, improved apparatus and methods are needed for placement of these types of systems.

Other catheter systems include semi-compliant angioplasty balloons which can provide moderate compliance in some lumen expansion applications. These balloons are only generally appropriate for peripheral vessel applications, however, and do not provide sufficient force to sufficiently expand and/or stent certain vessels including, for example, some coronary vessels. Moreover, these balloons may not provide optimal compliance in circumstances of high vessel curvature.

Other alternative stenting systems include the use of self-expanding stents such as nitinol-based stents, which can be expanded to a “memorized” diameter without requiring the use of a balloon for full expansion. However, self-expanding stents may also not provide sufficient radial force to properly retain the shape of some vessel walls such as in, for example, some coronary vessels.

Solutions are thus needed which allow for a balloon-expanded stent to be placed conformingly in bifurcated vessels or other tenuously shaped areas while retaining sufficient radial force within high-pressure vessels, and while minimizing the expense and risks of the procedure.

### BRIEF SUMMARY OF THE INVENTION

Aspects of the invention provide systems, procedures and apparatus for analyzing and treating body lumens, including highly curved vessels and vessel branches such as in, for example, a stent bifurcation procedure. In an embodiment of the invention, a system is provided including a catheter having a lumen-expanding balloon disposed about the catheter's distal end. In an embodiment of the invention, the balloon is deployed with a stent having a predetermined opening adapted to be highly conformant with a branch vessel opening. In another embodiment of the invention, the balloon is a pre-shaped balloon adapted to substantially conform with the curvature of a vessel.

The balloon catheter is integrated with one or more waveguides comprising at least one transmission output and at least one transmission input. The system can be programmed to gather information from the waveguides so as to direct the positioning, including rotational and/or longitudinal positioning, of the balloon and/or stent across a bifurcation and/or a highly curved vessel area. In an embodiment of the invention, the one or more transmission outputs and inputs are positioned to transmit and receive light about a section of the periphery of the shaped balloon. In an embodiment of the invention, the system is configured for providing information for positioning a pre-shaped balloon to conform with a vessel area upon expansion.

In an embodiment of the invention, waveguides are connected to a light source for distributing light radiation and connected to a detector for collecting light radiation about the balloon. The system can include one or more devices such as an intensity meter, a spectrometer, and/or an interferometer for analyzing the light radiation collected from outside the balloon wall. The one or more devices can be used to calculate and monitor the depth of blood between the balloon wall and the vessel wall and for positioning the balloon for optimal deployment.

The system can be configured to provide analysis through various wavelength ranges of radiation including, for example, visible and near-infrared radiation. An embodiment of the invention is configured to transmit and receive across wavelengths between about 200 and 2500 nanometers and, in a further embodiment of the invention, configured to transmit and receive across wavelengths of between about 300 and 700 nanometers. The system can be configured to transmit and receive across one or more single or multiple wavelength bands. In an embodiment of the invention, a range of one or more transmission wavelengths is distinct from a range of one or more detected wavelengths as in, for example, a fluorescence spectroscopy system. An embodiment of the invention includes transmitting through blood across one or more wavelengths centered about an excitation wavelength of, for example, about 450 nanometers and detecting a responsive emission across one or more wavelengths centered about, for example, 520 nanometers.

In an embodiment of the invention, a system is configured for estimating the distance between a section of the balloon's wall and a vessel wall in order to locate a branch vessel's opening with respect to the catheter.

In an embodiment of the invention, a stent with an expanded or “trap-door” opening, for example, can be positioned on a balloon to be subsequently deployed and positioned with respect to a branch vessel opening. The “trap-door” or expanded strut opening aligned with a branch vessel can be used, for example, to subsequently place an additional stent through the opening such as in a bifurcation procedure. In an embodiment of the invention, the information can be used to subsequently place a pre-shaped balloon across a highly curved area such that the pre-shaped balloon, in its expanded state, would align with the curvature of the vessel area. In an embodiment of the invention, the information about a vessel wall's proximity to the catheter can be used to determine the direction of curvature of the vessel area with respect to the catheter in order to place and conform a pre-shaped balloon within the vessel upon expansion.

In an aspect of the invention, a system is provided for treating a body lumen including a catheter having a flexible conduit that is elongated along a longitudinal axis and suitable for insertion into a body lumen, the conduit having a proximal end and a distal end. The system includes one or more waveguides integrated with the flexible conduit, the one or more waveguides constructed and arranged to deliver and collect radiation concentrated along a predetermined radial axis of the conduit, the predetermined radial axis of the conduit substantially aligned with respect to at least one therapy delivery component of the catheter. The system also includes at least one radiation source connected to a transmission input of the one or more waveguides integrated with the flexible conduit and at least one optical detector connected to a transmission output of the one or more waveguides integrated with the flexible conduit.

In an embodiment, the system includes an expandable balloon about the distal end of the conduit, wherein the at least one therapy delivery component includes a feature of an angioplasty catheter.

In an embodiment, the feature of the angioplasty catheter includes a stent. In an embodiment, the feature of said angioplasty catheter includes a predetermined opening within said stent.

In an embodiment, the feature of the angioplasty catheter includes an expandable balloon. In an embodiment, the feature of the angioplasty catheter includes a predetermined preformed portion of the expandable balloon.

In an embodiment, the system includes a controller programmed to process data from the optical detector so as to direct an alignment of the at least one therapy delivery component.

In an embodiment of the invention, a system includes an analysis subsystem programmed and configured for determining a relative measure of blood depth outward along the predetermined radial axis from the conduit. In an embodiment of the invention, a radiation source is configured to supply radiation of one or more wavelengths within the range of about 250 to 2500 nanometers. In an embodiment of the invention, the radiation source is configured to supply radiation of one or more wavelengths within the range of about 400 and 1400 nanometers. In an embodiment of the invention, the radiation source is configured to supply radiation of one or more wavelengths within the range of about 400 and 700 nanometers.

In an embodiment of the invention, a radiation source is configured and arranged to supply radiation of one or more predetermined wavelengths and wherein the optical detector is configured and arranged to selectively detect radiation distinct from wavelengths supplied by the radiation source. In an embodiment of the invention, the system includes a dichroic filter arranged to separate radiation of wavelengths selected for delivery and radiation of wavelengths selected for collection and detection.

In an embodiment of the invention, a radiation source and optical detector are configured and arranged to induce and detect fluorescence. In an embodiment of the invention, the radiation source is configured to supply radiation including wavelengths of less than about 500 nanometers and the optical detector is configured and arranged to selectively detect radiation of greater than about 500 nanometers. In an embodiment of the invention, the radiation source is configured to supply radiation including a wavelength of 450 nanometers and wherein the optical detector is configured and arranged to selectively detect radiation including a wavelength of 520 nanometers.

In an embodiment of the invention, the system includes an optical arrangement for supplying and collecting radiation through a combined delivery output and collection input.

In an embodiment of the invention, an optical detector is connected to a spectrometer. In an embodiment of the invention, the spectrometer is configured to perform spectroscopy selected from the group of spectroscopy methods including fluorescence, light scatter, optical coherence reflectometry, optical coherence tomography, speckle, correlometry, Raman, and diffuse reflectance spectroscopy.

In an embodiment of the invention, a radiation source and an optical detector are connected to an interferometer.

In an embodiment of the invention, the system includes an intensity meter for measuring the level of signal associated with a characteristic of bodily blood or tissue. In an embodiment of the invention, the characteristic of bodily blood or tissue includes the depth of blood across an area of interest.

In an embodiment of the invention, the system includes a control and display device. In an embodiment of the system, the control and display device includes an indicator of blood-depth signal intensity to an operator. In an embodiment of the invention, the control and display device includes a mechanism for controlling the rotational position of the flexible conduit. In an embodiment of the invention, the control and display device is hand-held.

In an aspect of the invention, a catheter for placement within a body lumen is provided. The catheter includes a flexible conduit that is elongated along a longitudinal axis, the flexible conduit having a proximal end and a distal end. The catheter further includes at least one therapy delivery component and one or more waveguides positioned along the flexible conduit. The one or more waveguides are constructed and arranged to deliver and collect radiation concentrated about a predetermined radial axis of the conduit, the predetermined radial axis substantially aligned with respect to the at least one therapy delivery component.

In an embodiment of the invention, the therapy delivery component comprises a predetermined opening of a stent. In an embodiment of the invention, the predetermined opening is formed to substantially conform with an opening of a vessel bifurcation so as to reduce the impedance of blood flow. In an embodiment of the invention, the predetermined opening is positioned between the longitudinal ends of the stent body. In an embodiment of the invention, the predetermined opening forms an extended circumferential gap. In an embodiment of the invention, the predetermined opening is positioned at a longitudinal end of the stent body. In an embodiment of the invention, the predetermined opening forms a beveled end out of the stent body.

In an embodiment of the invention, an at least one waveguide consists of a single waveguide constructed and arranged to simultaneously deliver and collect radiation.

In an embodiment of the invention, an at least one waveguide includes at least one delivery waveguide and at least one separate collection waveguide.

In an embodiment of the invention, the catheter includes an expandable balloon about the distal end of the conduit in which a feature of the expandable balloon is a therapy delivery component. In an embodiment of the invention, a therapy delivery component of the balloon includes a pre-formed area of the balloon configured to dilate an adjacent opening of a vessel bifurcation. In an embodiment of the invention, this pre-formed area forms a bulbous augmentation of the balloon when expanded. In an embodiment of the invention, a therapy delivery component of the balloon causes the balloon to bend along its longitudinal axis when expanded so as to improve conformance of the expanded balloon within the shape of a curved vessel.

In an aspect of the invention, a method for treatment of a body lumen is provided. The methods include the step of inserting into a body lumen a catheter including a flexible conduit having at least one therapy delivery component. The flexible conduit includes one or more waveguides positioned along the flexible conduit, the one or more waveguides constructed and arranged to deliver and collect radiation concentrated about a predetermined radial axis of the conduit, the predetermined circumferential position substantially aligned with respect to at least one therapeutic component. The method further includes the steps of maneuvering the conduit into a designated region of the body lumen designated for treatment and optimizing rotational alignment of the at least one therapeutic component for providing therapy within the body lumen. The step of optimizing rotational alignment includes repeating the steps of rotating the flexible conduit within the body lumen, measuring and analyzing optical signals collected through the one or more waveguides, and relating the analysis of the optical signals with an optimal rotational position. The method further includes a step of activating the therapeutic component.

In an embodiment of the invention, the designated region of the body designated for treatment includes a vessel bifurcation. In an embodiment of the invention, the at least one therapeutic component includes a stent with a predetermined opening. In an embodiment of the invention, the step of optimizing rotational alignment of the conduit optimizes alignment of the opening of the stent with the opening of the vessel bifurcation.

In an embodiment of the invention, the designated region of the body designated for treatment includes a vessel area highly curved along its longitudinal axis. In an embodiment of the invention, the at least one therapeutic component comprises an expandable balloon manufactured to become curved upon expansion so that it substantially conforms to the longitudinal curvature of the vessel area. In an embodiment of the invention, the step of optimizing rotational alignment of the conduit optimizes rotational orientation of the balloon to longitudinally conform with the highly curved vessel.

In an embodiment of the invention, an at least one therapeutic component comprises an expandable balloon having a pre-formed area configured to dilate an adjacent opening of a vessel bifurcation upon expansion. In an embodiment of the invention, the step of optimizing rotational alignment of the conduit optimizes rotational orientation of the balloon to align the pre-formed area with the adjacent opening.

In an embodiment of the invention, a step of measuring and analyzing optical signals comprises delivering and collecting radiation concentrated along a predetermined radial axis of the conduit. In an embodiment of the invention, the step of measuring and analyzing optical signals comprises measuring a signal associated with a blood depth spanning radially outward along the predetermined radial axis of the conduit. In an embodiment of the invention, the signals associated with a blood depth are analyzed at a plurality of catheter rotations to distinguish between a vessel bifurcation opening and a lack of an opening along the predetermined radial axis of the conduit. In an embodiment of the invention, the signals associated with a blood depth are analyzed at a plurality of catheter rotations to distinguish between a relatively convex shaped vessel wall and a relatively concave shaped vessel wall about the predetermined radial axis of the conduit.

In an embodiment of the invention, a step of activating the therapy delivery component comprises expanding a lumen expanding balloon.

In an embodiment of the invention, a step of measuring and analyzing optical signals comprises delivering radiation of wavelengths within the range of about 250 to 2500 nanometers. In an embodiment of the invention, the step of measuring and analyzing optical signals comprises delivering radiation of wavelengths within the range of about 400 to 1400 nanometers. In an embodiment of the invention, the step of measuring and analyzing optical signals comprises delivering radiation of wavelengths within the range of about 400 to 700 nanometers.

In an embodiment of the invention, a step of measuring and analyzing optical signals collected through the one or more waveguides comprises inducing and measuring fluorescence by delivering radiation of one or more wavelengths so as to induce fluorescence, and measuring the intensity of radiation generated from the fluorescence. In an embodiment of the invention, an at least one wavelength of the radiation measured from the fluorescence is distinct from the one or more wavelengths of the radiation delivered to induce fluorescence. In an embodiment of the invention, the one or more wavelengths of the radiation generated to induce fluorescence includes a wavelength of 450 nanometers and wherein the at least one wavelength of the radiation generated from the fluorescence includes a wavelength of 520 nanometers.

In an embodiment of the invention, a step of measuring and analyzing optical signals comprises performing spectroscopy selected from the group of spectroscopy methods consisting of fluorescence, light scatter, optical coherence reflectometry, optical coherence tomography, speckle, correlometry, Raman, and diffuse reflectance spectroscopy.

In an embodiment of the invention, a step of activating the therapeutic component comprises delivering therapeutic radiation to a targeted area.

In an embodiment of the invention, a step of longitudinally aligning the flexible conduit includes the steps of measuring and analyzing optical signals collected through the one or more waveguides, and relating the analysis of the optical signals with an optimal longitudinal position. In an embodiment of the invention, the step of longitudinally aligning the flexible conduit includes a plurality of steps of longitudinally moving the flexible conduit interspersed with a plurality of steps of measuring and analyzing optical signals collected through the one or more waveguides.

In an aspect of the invention, a method for treatment or analysis of a body lumen is provided. The method includes a step of inserting into a body lumen a catheter including a flexible conduit having at least one analysis or therapeutic component. The flexible conduit includes one or more waveguides positioned along the flexible conduit in which the one or more waveguides are constructed and arranged to deliver and collect radiation concentrated about a predetermined radial axis of the conduit and in which the predetermined radial axis is substantially aligned relative to at least one analysis component or therapy delivery component. The method further includes the steps of maneuvering the conduit into a designated region of the body lumen designated for analysis or treatment and optimizing positional alignment of the at least one analysis or therapeutic component within the body lumen. The step of optimizing positional alignment includes repeating the steps of moving the flexible conduit within the body lumen, measuring and analyzing optical signals collected through the one or more waveguides, and relating the analysis of the optical signals with an optimal position. The method further includes the step of activating the analysis component or therapy delivery component.

In an embodiment of the invention, a step of activating the at least one analysis or therapeutic component includes performing spectroscopy selected from the group of spectroscopy methods consisting of fluorescence, light scatter, optical coherence reflectometry, optical coherence tomography, speckle, correlometry, Raman, and diffuse reflectance spectroscopy.

In an embodiment of the invention, a step of optimizing positional alignment includes optimizing rotational alignment.

In an embodiment of the invention, a step of optimizing positional alignment includes optimizing longitudinal alignment.

### BRIEF DESCRIPTION OF THE DRAWINGS

The foregoing and other objects, features, and advantages of the invention will be apparent from the more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A are illustrative views of a catheter's distal and proximate ends, and a hand-held control and display device, in accordance with an embodiment of the invention.

FIG. 1B is a schematic block diagram illustrating an instrument for analyzing and medically treating a lumen, according to an embodiment of the present invention.

FIG. 2A is an illustrative schematic view of the distal and proximal ends of a balloon catheter deployed in a vessel bifurcation in accordance with an embodiment of the invention.

FIG. 2B is an expanded illustrative view of a section of the catheter shown in FIG. 2A including the terminating ends of transmission and collection fibers in accordance with an embodiment of the invention.

FIG. 2C is an illustrative cross-sectional view of the catheter shown in FIGS. 2A-2B.

FIG. 3A is an illustrative view of an angioplasty catheter with an expanded side-opening shown positioned across from a branch vessel according to an embodiment of the invention.

FIG. 3B is an illustrative view of an angioplasty catheter shown positioned within branch vessel and passing through the expanded opening of the stent of FIG. 3A according to an embodiment of the invention.

FIG. 4A is an illustrative view of an angioplasty catheter having a stent positioned immediately past the opening of a branch vessel in accordance with an embodiment of the invention.

FIG. 4B is an illustrative view of a catheter with a bevel-ended stent is shown positioned within a branch vessel according to an embodiment of the invention.

FIG. 5A is a simplified schematic showing an optical component layout for measuring blood volume from a catheter system in a single fiber in accordance with an embodiment of the invention.

FIG. 5B is an illustrative view of a catheter system incorporating the component layout of FIG. 5A.

FIG. 6A is a simplified schematic of an optical component layout for measuring blood volume from a multiple-fiber catheter system in an embodiment of the invention.

FIG. 6B is an illustrative view of a catheter system incorporating the components of FIG. 6A.

FIG. 7A is a chart of a study conducted comparing known depths of a blood medium with diffuse reflectance spectral absorbance measurements taken through the blood medium.

FIG. 7B is a chart compiling various spectra peaks associated with water and blood media relevant to large and small vessel diameter ranges.

FIG. 8A is an illustrative view of a single transmission/collection fiber integrated with the distal end of a balloon catheter in an embodiment of the invention.

FIG. 8B is a side perspective view of the single-fiber embodiment of the invention shown in FIG. 8A with a stent crimped about the distal end of a catheter.

FIG. 8C is an illustrative cross-sectional view of the single fiber embodiment shown in FIGS. 8A-8B.

FIG. 9A is an illustrative side-perspective view of the distal end of a balloon catheter having single transmission/collection fiber terminated with a prism redirector in an embodiment of the invention.

FIG. 9B is an illustrative head-on perspective view of the embodiment shown in FIG. 9A.

FIG. 9C is an illustrative head-on perspective view of the distal end of a balloon catheter having transmission and collection collection fibers terminated with a prism redirector in an embodiment of the invention.

FIG. 10A is an illustrative view of the distal end of a balloon catheter having a transmission and collection fiber arranged with a cone-shaped redirecting element in an embodiment of the invention.

FIG. 10B is an illustrative view of the distal end of a balloon catheter having a cone-shaped redirecting element positioned adjacent the proximal end of a balloon and crimped stent in an embodiment of the invention.

FIG. 11A is an illustrative view of a dual-fiber embodiment of the invention with fibers arranged on the outside of a balloon.

FIG. 11B is an illustrative view of the dual-fiber embodiment of FIG. 11A including a crimped stent.

FIG. 12A is an illustrated view of an embodiment of the invention including a distal end of a catheter having a pre-shaped balloon and a stent with an expanded opening.

FIG. 12B is an illustrated view of the catheter of FIG. 12A with its pre-shaped balloon in an expanded state.

FIGS. 12C-12D are illustrated views of the distal end of FIGS. 12A-12B deployed in a vessel bifurcation, with its pre-shaped balloon in, respectively, unexpanded and expanded states.

FIGS. 13A and 13B are illustrative views of a balloon catheter's distal end with a pre-shaped balloon in, respectively, unexpanded and expanded states according to an embodiment of the invention.

FIG. 13C is an illustrative view of the balloon catheter of FIGS. 13A-B including a crimped stent being rotationally positioned within a curved vessel area.

FIG. 13D is an illustrative view of the balloon catheter and stent of FIG. 13C shown expanded within a curved vessel area.

FIGS. 14A-14B are illustrative views of a balloon catheter having multiple balloons for conformant placement in a curved vessel in, respectively, unexpanded and expanded states according to an embodiment of the invention.

FIG. 14C is an illustrative view of the balloon catheter of FIGS. 14A-B including a crimped stent being rotationally positioned within a curved vessel according to an embodiment of the invention.

FIG. 14D is an illustrative view of the balloon catheter of FIG. 14C in an expanded state within a curved vessel area.

### DETAILED DESCRIPTION OF EMBODIMENTS OF THE INVENTION

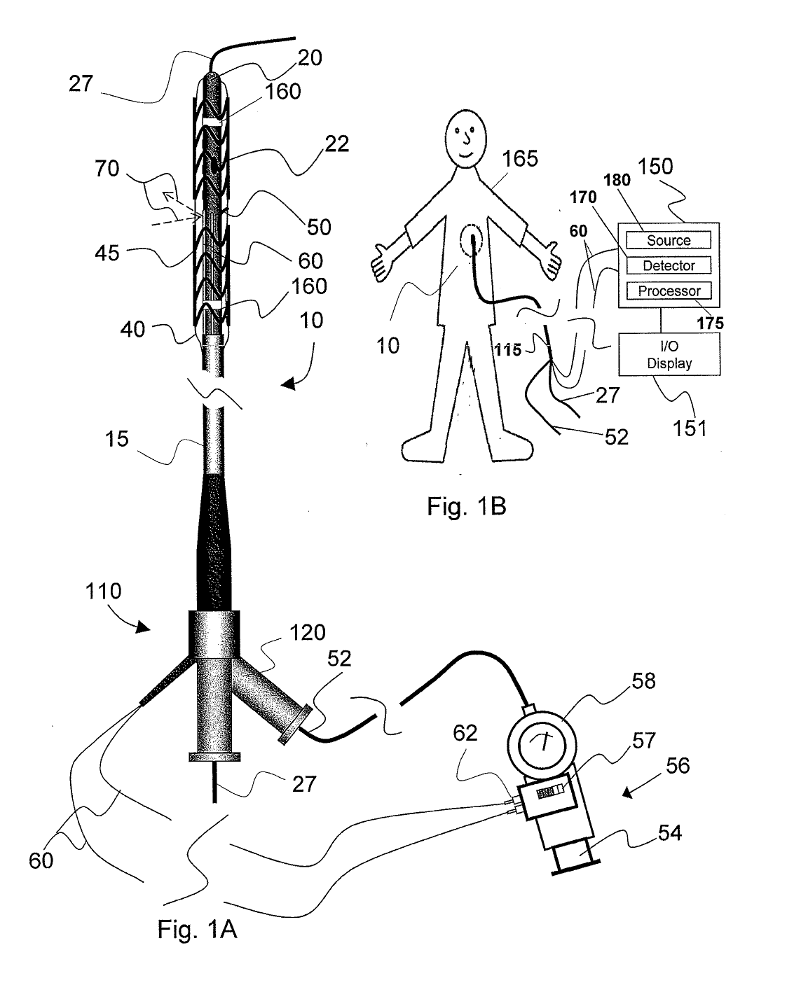

Referring to FIG. 1A, an illustrative view is shown of a catheter's distal and proximate ends in accordance with an embodiment of the invention. A catheter 10 includes a catheter body 20 and catheter sheath 15 about which an expandable balloon 40 is bound such as in accordance with, for example, an angioplasty balloon catheter, which can be used for stenting, pre-stenting dilation and/or pre-stenting analysis. A flush port 22 allows for fluid media (e.g., saline) to expand balloon 40. Radiopaque marker bands 160 aid in locating and placement of the catheter 10 within a lumen such as with, for example, a fluoroscope. In an embodiment of the invention, a stent 45 is crimped about balloon 40 for purposes of subsequent deployment in a vessel such as in a percutaneous transluminal angioplasty procedure. Stent 45 includes an expanded opening 50 designed to conform with an opening of a branch vessel. Fibers 60 terminate along expanded opening 50 for transmitting and collecting radiation (e.g., along sample trace lines 70) about an area adjacent expanded opening 50. Fibers 60 lead to a device 56 and are connected through connectors 62. Device 56 is used for manipulating or controlling the catheter 10, supplying and detecting radiation transmitted through fibers 60, processing signals from detected radiation, and displaying processed data to an operator. As described in additional detail in further embodiments of the present invention, processed data from detected radiation can be used to guide the longitudinal and rotational position of opening 50 in correspondence with a vessel branch opening.

The proximal end of the catheter 10 includes a section 110 through which fibers 60, a guidewire 27, and an inflation media supply line 52 are integrated. The proximate ends of fiber lines 60 are connected to the device 56. Device 56 can include a source and detector as described in additional detail in further embodiments included herein. A display 58 is provided for relaying information (e.g., the intensity of detected signals) to an operator of the device. Device 56 additionally includes a knob 54 for controlling the supply of inflation media to balloon 40.

Referring further to FIG. 1B, a schematic block diagram illustrating an instrument, such as an analysis device 150 for analyzing and medically treating a lumen is shown deployed in a patient 165 according to an embodiment of the present invention. An analysis device 150 such as, for example, an intensity meter, an interferometer, and/or spectrometer is connected through fibers 60 and can process and analyze light gathered from areas about the balloon such as, for example, blood and tissue. A source 180 and detector 170 are integrated with device 150 for the distribution and collection of radiation. The device includes a processor 175 for coordinating source 180 and detector 170 signals and processing data (e.g., spectroscopic data) for transfer, display, and/or further analysis. In an embodiment, an LED display 57 indicates the intensity of a signal such as, for example, signals in relation to the amount of blood detected through fibers 60. Analysis of light about the balloon can provide information about the geometry of vessels within which the balloon is located such as, for example, the distance between a portion of the balloon wall and the nearest vessel wall. Methods of analysis include, for example, Raman spectroscopy, infrared spectroscopy, fluorescence spectroscopy, optical coherence reflectometery, optical coherence tomography, diffuse-reflective spectroscopy, near-infrared spectroscopy, and/or low-coherence interferometry. As well as in, for example, copending, related U.S. Patent Publication No. 2007/0078500 A1 by Ryan et al. (“Ryan '500”), additional methods can be applied as described in U.S. Patent Publication No. US 2006/0024007 A1 by Carlin, et al., the contents of each of which is herein incorporated by reference in its entirety. Configuration and control of device 150 and the output of results can be performed through an I/O control and display device 151.

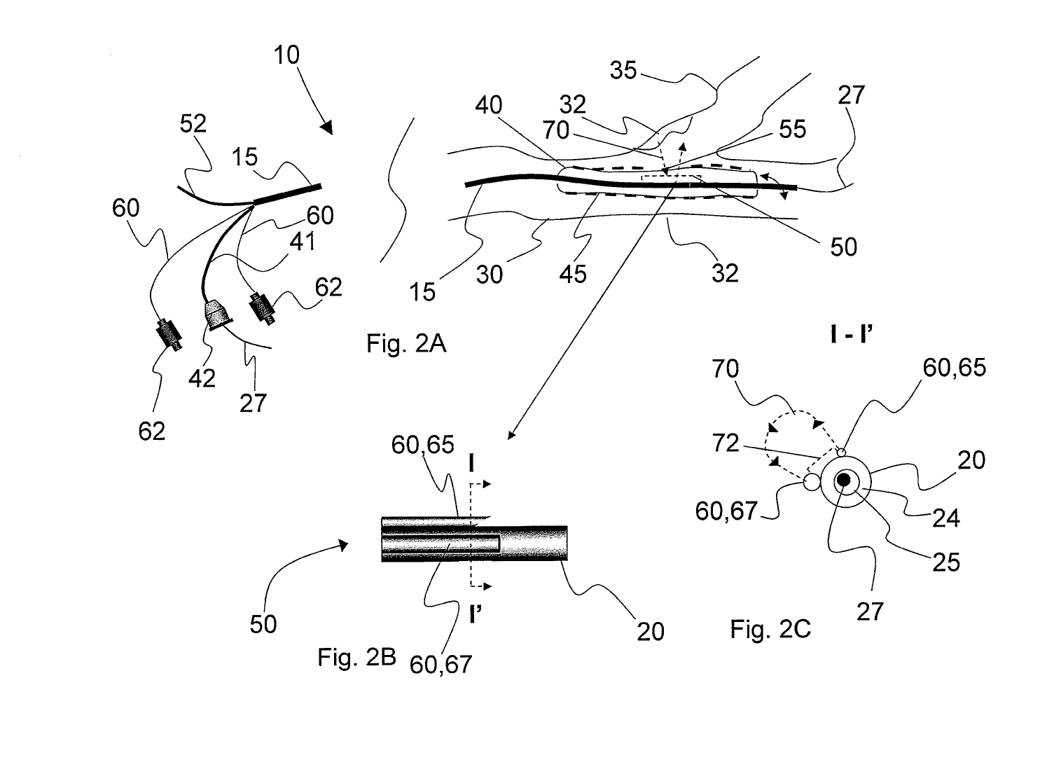

FIG. 2A is an illustrative schematic view of the distal and proximal ends of a balloon catheter deployed in a vessel in accordance with an embodiment of the invention. FIG. 2B is an expanded illustrative view of a section of the catheter shown in FIG. 2A including the terminating ends of transmission and collection fibers in accordance with an embodiment of the invention. FIG. 2C is an illustrative cross-sectional view of a section 50 of the catheter shown in FIGS. 2A-2B across line I′-I″ of FIG. 2B.

Referring to FIGS. 2A-2C, a catheter 10 includes a catheter body 20 and catheter sheath 15 about which an expandable balloon 40 is bound such as in accordance with, for example, an angioplasty balloon catheter. A stent 45 is crimped about the balloon 40 such that, upon expansion of balloon 40, stent 45 can be deployed within a vessel. A source fiber 65 and collection fiber 67 are integrated with catheter 10, and pass along catheter body 20 with connector ends 62 connected to the source fiber 60, and extend from the proximate end of catheter sheath 15. The intervening area between catheter body 20 and a guidewire lumen 25 provides a flush lumen 24 for the transfer of fluid media to and from balloon 40. A guidewire 27 may be placed through port 42 and guidewire lumen 41 for initially directing the positioning of catheter 10 such as in a percutaneous transluminal angioplasty procedure.

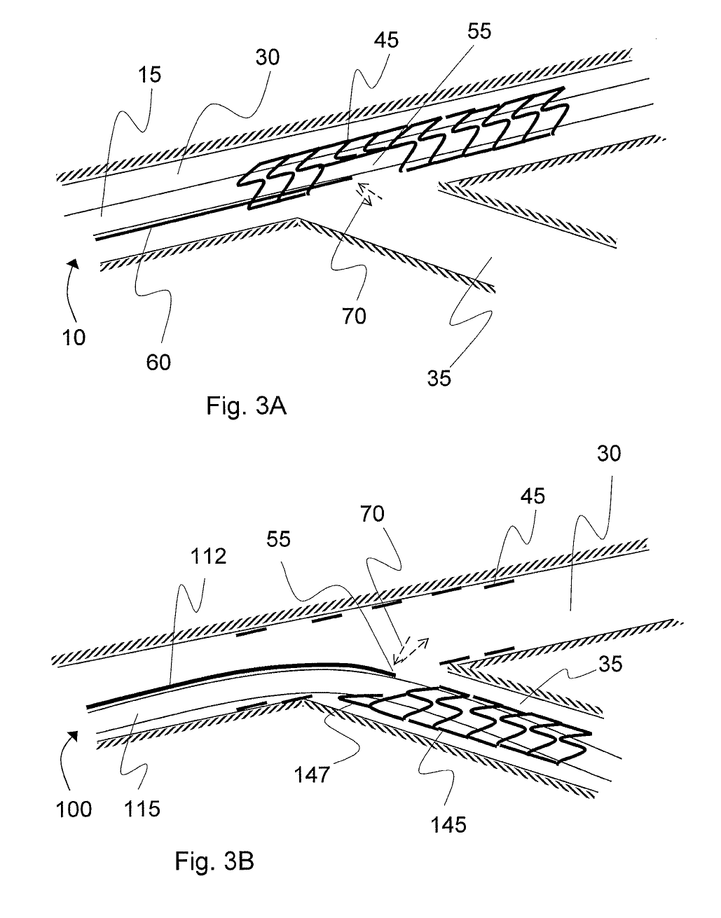

Section 50 of catheter 10 includes the distribution and collection ends, respectively, of one or more fibers such as, for example, source fiber 65 and collection fiber 67, which are fixed adjacent to catheter body 20 within balloon 40 so they can distribute and collect light about the outside of balloon 40. Balloon 40 is manufactured to be optically clear to the selected radiation and can be manufactured with various materials including, for example, nylon, polyethylene, or other translucent polymers. Stent 45 includes an expanded opening 55 through which a subsequent stent (e.g., see FIG. 3B) could be passed such as in a bifurcation procedure. The expanded opening 55 also allows for radiation to more easily pass unblocked to and from fibers 65 and 67.

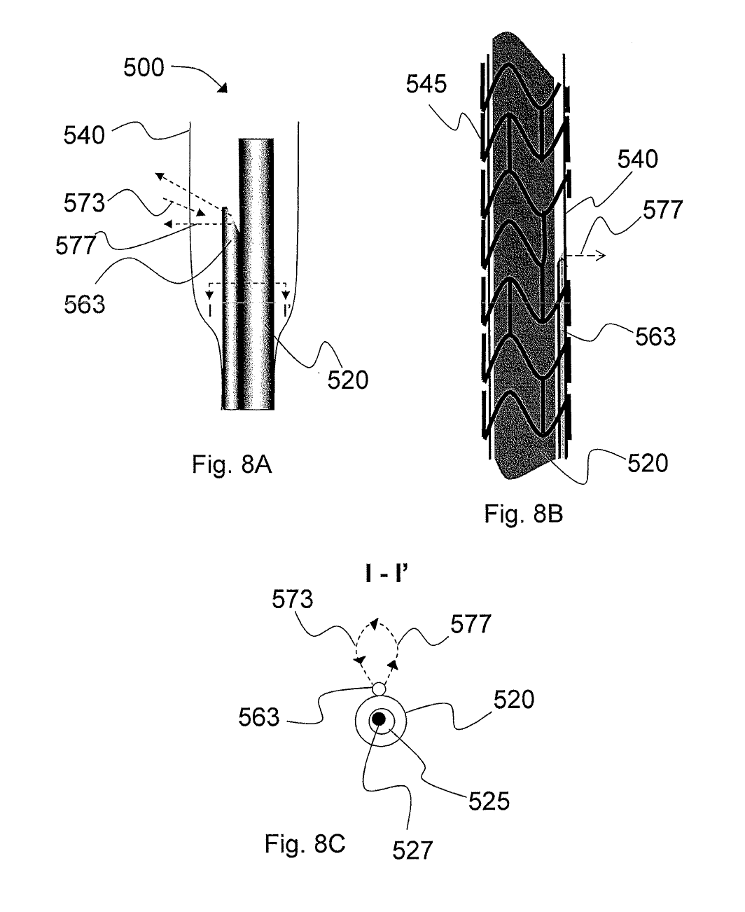

Referring in particular to FIG. 2C, light distributed from fiber 65 is shown traveling along a sample path 70 to collection fiber 67. The terminating ends of fibers 65 and 67 can be positioned, shaped and/or surfaced according to various embodiments to distribute and collect light in a predetermined manner as described in further embodiments below (see, e.g., FIGS. 8-11 and accompanying descriptions). The positions of terminating ends of fibers 65 and 67 are aligned with respect to other components of catheter 10 and, in an embodiment of the invention, aligned to aid in providing information about the relative orientation of balloon 40 and other components including, for example, the orientation of opening 55 of stent 45. For example, a reading from radiation collected through fiber 67 can indicate the depth of blood between a location on catheter 10 and a vessel side-wall. When catheter 10 is oriented such that fibers 65 and 67 get a maximal depth reading (i.e., they are optimally positioned adjacent to the opening of branch vessel 35), stent 45 can be expanded so that expanded opening 55 optimally aligns with the opening of branch vessel 35. In other embodiments of the invention, multiple distribution and collection fibers can be used or a single fiber can be used for both distribution and collection of radiation.

In an embodiment of the invention, the separation distance 72 between the distal ends of fibers 65 and 67 and their numerical aperture are optimized with respect to the diameter of catheter 10 and the expected diameter of a vessel in which the catheter is deployed. Both separation distance 72 and numerical aperture will generally influence the direction and depth of signals traveling to and from the catheter. Numerical aperture and separation distance may also affect the breadth of the tissue surface area analyzed in each measurement, which should preferably be minimized for purposes of accurate positional determination. The diameter of fibers 65 and 67 should also be minimized (e.g. distribution fiber 65 is of less than about a 100 micron diameter and collection fiber 67 is of less than about a 200 microns diameter) so that the catheter can remain as flexible as possible. Optimum separation distances and numerical apertures can be characterized through tests of signals through anticipated depths of blood/tissue media. A larger numerical aperture will generally be required of a collection fiber in order to facilitate the loss of signal strength between transmission and collection. The separation distance is also be limited by the amount of power that surrounding tissue can safely withstand from a radiation source, which should generally be limited to a maximum of about 20 milliwatts.

Referring to FIG. 3A, an illustrative view of an angioplasty catheter 10 with a stent 45 having an extended opening 55 is shown positioned across from a branch vessel 35 according to an embodiment of the invention. Referring also to FIG. 3B, a catheter 100 is shown positioned within branch vessel 35 passed through expanded opening 55 of deployed stent 45. Catheter 100 includes one or more fibers 112 along catheter sheath 115 that terminate closely to the proximate end of a bevel-ended stent 145. Stent 145, shown crimped about catheter 100, includes a beveled end 147 so it can be obliquely positioned along the intersection between main vessel 30 and branch vessel 35, thus minimizing the amount material unnecessarily protruding into the blood flow path of the bifurcation. Excessive blockage of flow can lead to, for example, thrombosis and other serious conditions. One or more fibers 112 are positioned to distribute and collect light in order to provide analysis and guidance of an optimal rotation of catheter 100 and of the beveled end 147 of stent 145 with respect to the bifurcation. Deployment of stent 145 will thus result in a stenting of a bifurcation between vessel 30 and branch vessel 35 which provides substantially reduced levels of obstructive material as compared to traditional bifurcation procedures.

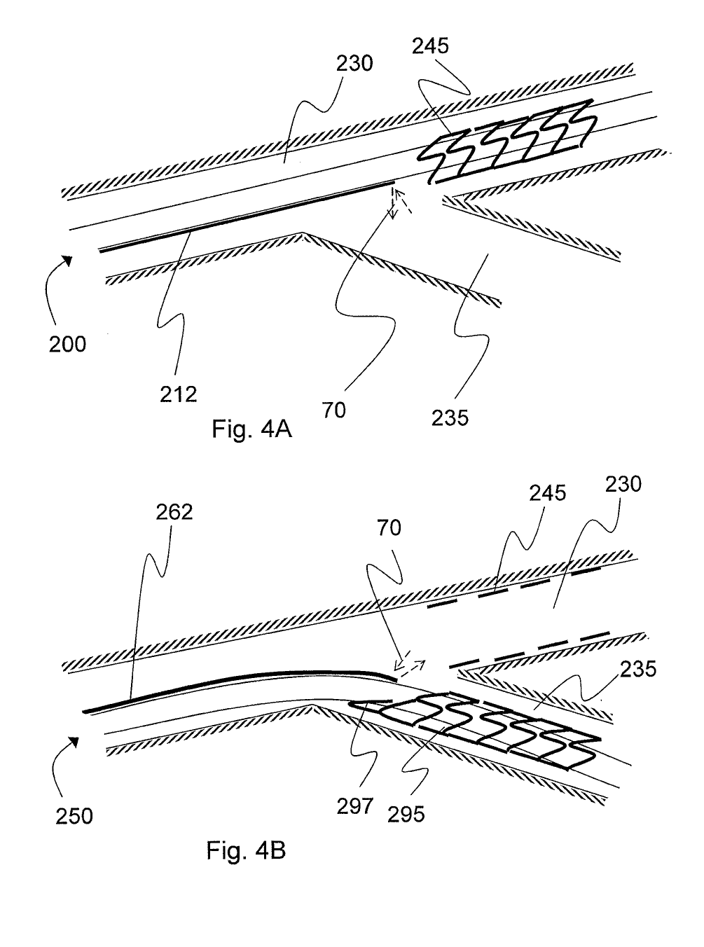

Referring to FIG. 4A, an illustrative view of an angioplasty catheter 200 is shown having a stent 245 positioned immediately past the opening of a branch vessel 235 in accordance with an embodiment of the invention. One or more fibers 212 are arranged with probe ends near the proximal end of stent 245 in order to help guide and position catheter 200 such that the proximal end of stent 245 is as close as possible to the branch vessel opening 235 without blocking blood flow through branch vessel 235.

Referring to FIG. 4B, a catheter 250 with a bevel-ended stent 295 is shown positioned within branch vessel 235 according to an embodiment of the invention. One or more fibers 262 are arranged close to the proximal end of stent 295. Fibers 262 can be used in accordance with embodiments described herein to help guide the longitudinal and rotational position of stent 295 and its beveled end 297 with respect to the opening of branch vessel 235. Beveled end 297 can thus be positioned obliquely with the opening of branch vessel 235 so as to help minimize unnecessary obstruction of vessel 230.

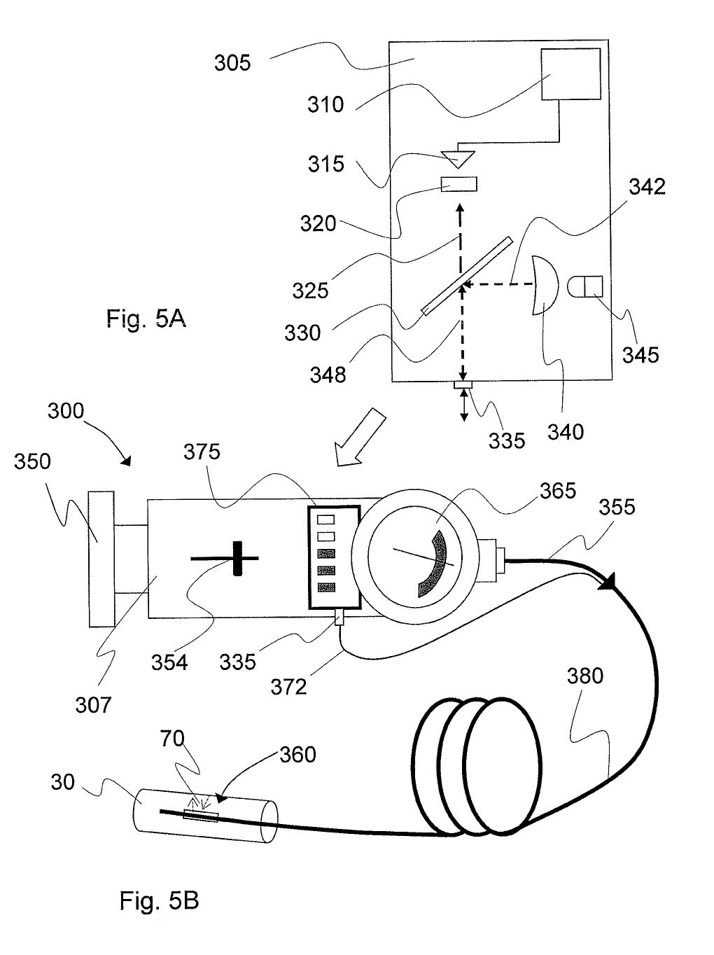

Referring to FIG. 5A, a simplified schematic shows an optical component layout 305 and light transmission paths for integration with an enclosure 307 and catheter system 300 (shown in FIG. 5B) for measuring blood volume in a single fiber embodiment of the invention. Referring also to FIG. 5B, an illustrative view of catheter system 300 is shown which can incorporate the components of layout 305. Optical component layout 305 includes a source 345 directing radiation through a focus lens 340 and along path 342 to a filter 330. Source 345 can be, for example, an LED or laser device. Filter 330 reflects radiation of a selected wavelength range along path 348 and to a connector interface 335 connected to a fiber 372. Fiber 372 extends through a catheter sheath 380 to the distal end 360 of catheter system 300 such as, for example, in accordance with the embodiment of FIGS. 8A-8C and accompanying description. Sample paths 70 illustrate radiation distributed and collected through fiber 372 in an embodiment of the invention.

Collected radiation, e.g. fluorescence radiation, may then travel along path 348 to filter 330. In an embodiment of the invention, filter 330 can be selected to be transmissive to a wavelength range of interest different from an excitation-inducing wavelength range, such as 30 to 100 nm longer than an excitation-inducing wavelength range. Radiation passing through filter 330 then travels along path 325 to a photo sensor 320 capable of measuring the intensity of the selected wavelength range. In an embodiment of the invention, the radiation wavelength range produced by source 345 is selected to cause an excitation of a different wavelength range within the targeted medium (i.e., blood). Fluorescence filters and other filters for separating wavelength ranges are available from a variety of commercial vendors including, for example, Semrock, Inc. of Rochester, N.Y.

In an embodiment of the invention, a source wavelength range can be between about 200 and about 2500 nanometers. In a further embodiment, a source wavelength range can be between about 300 and 1400 nanometers. In a further embodiment, a source wavelength range can be between about 400 and 700 nanometers. In an embodiment, an excitation-inducing wavelength of about 450 nanometers produces a fluorescence excitation emission wavelength in blood of about 520 nanometers. Source 345 can be a low-cost LED which is selected to provide a wavelength range between, for example, about 400 and 500 nanometers, concentrating energy at about 450 nanometers. Filter 330 can be selected, for example, to reflect radiation greater than about 500 nanometers including 520 nanometer radiation. Upon consideration of the present disclosure, various modified arrangements of filters, sources, and other optical components, optical paths, and wavelength ranges would be apparent to one of ordinary skill in the art.

A fluid supply line 355 and fiber 372 are integrated into a catheter sheath 380 leading to an expandable balloon assembly 360 (shown within a vessel area 30) such as in accordance with various embodiments of the present invention disclosed herein. An operator can, for example, rotate the distal end assembly 360 to various positions interspersed in between steps of performing optical analysis. Rotation of distal end assembly 360 and analysis of lumen area 30 can be performed in accordance with various embodiments of the invention including, for example, those disclosed in connection with FIGS. 2-4 and FIGS. 12-14.

A signal processor 315 translates a reading from sensor 320 to a signal to be used with an I/O and/or display device 310 such as an intensity indicator 375, which can indicate to an operator the relative depth of blood of an area adjacent a pre-determined portion of the distal end of the catheter system 300. Intensity indicator 375 can be comprised of one or more LEDs, for example, in which the one or more of the LEDs indicate the level of depth via states of on or off and/or varying intensity. In another embodiment of the invention, an audio signal generator (not shown) is integrated into the system 305 to indicate depth via tones and/or volume. An inflation lever 350 controls the distribution of inflation media within a balloon of an expandable balloon assembly 360. A pressure indicator 365 displays the amount of pressure within the balloon. In an embodiment of the invention, an intensity lever and/or amplification control lever 354 is optionally included for purposes controlling and/or calibrating the sensitivity of indicator 375 such as by adjusting the intensity of source radiation from source 345 or the amplification level of the collected signal through photo-sensor 320. Calibrating sources/signals may be useful depending on the type and size of a targeted treatment area.

In an embodiment of the invention, catheter system 300 and layout 305 is manufactured for disposable cost-effective use. For example, the enclosure 307 can be molded of easily assembled plastic components including its movable parts such as, for example, balloon media supply knob 354, source intensity control knob 350, among other various parts. Media fluid pressure indicator can be of a common type used in other angioplasty catheters. Intensity indicator 375 can be a simple LED-type indicator calibrated to reflect a general relative intensity output from a signal processor such as processor 315. Various filters and other optical components of layout 305 can also be made of low-cost plastic parts such as, for example, filter 330 and focusing lens 340. Source 345 can be powered by a low-cost disposable/replaceable battery (not shown) housed in enclosure 307.

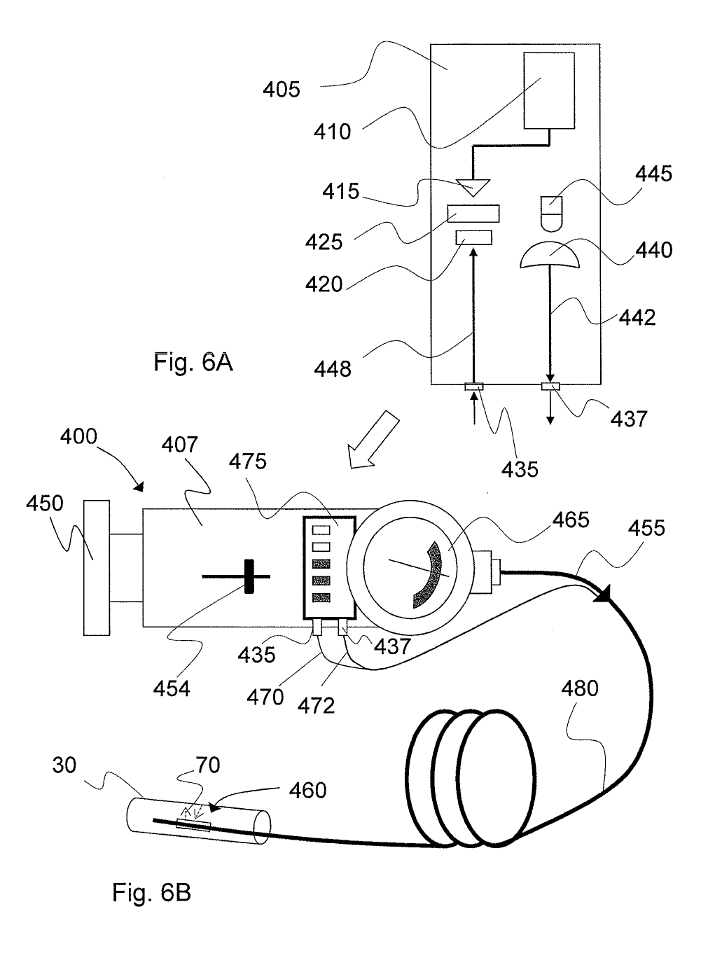

Referring to FIG. 6A, a simplified schematic shows an optical component layout 405 for integration within a catheter system 400 for measuring blood depth in a dual-fiber embodiment of the invention. Referring also to FIG. 6B, an illustrative view of the catheter system 400 incorporating the components 405 of FIG. 6A is shown. Optical component layout 405 includes a source 445 directing radiation along path 442 to an output connector 437 and fiber 472. Source 445 can be, for example, an LED or laser device and can include a focusing element 440 in order to more precisely concentrate and/or direct radiation. Fiber 472 extends through a catheter sheath 480 to the distal end 460 of catheter system 400. Distal end 460 can include, for example, a balloon assembly such as in accordance with various multiple-fiber embodiments disclosed herein.

Radiation is collected through the distal end of fiber 470 (integrated in the distal end 460 of catheter system 400) and transmitted through input connector 435. Collected radiation may then travel along a sample path 448 to a photo sensor 420. In an embodiment of the invention, an intensity inverter 425 inverts the signal received from sensor 420 in order to provide an absorbance signal to a signal processor 415 and to an I/O and/or display device 410 for supplying data to an operator or externally connected device. In an embodiment of the invention, absorbance data is used to provide diffuse-reflectance spectroscopic analysis of surrounding blood and tissue such as for calculating a measurement of the span of blood between a predetermined location on distal end 460 and a vessel wall.

An operator can rotate the distal end assembly 460 to various positions while providing analysis during a procedure such as in accordance with various embodiments of the invention (e.g., FIGS. 2-4 and FIGS. 12-14). An indicator 475 can indicate data analysis results (e.g., the relative span of blood adjacent from the probe) to an operator. An inflation knob 450 controls the volume of inflation media within a balloon of an expandable balloon assembly 460. A pressure indicator 465 displays the amount of pressure within the balloon. The catheter system 400 and component layout 405 can be manufactured with generally low-cost disposable components in a similar manner as that described in reference to catheter system 300.

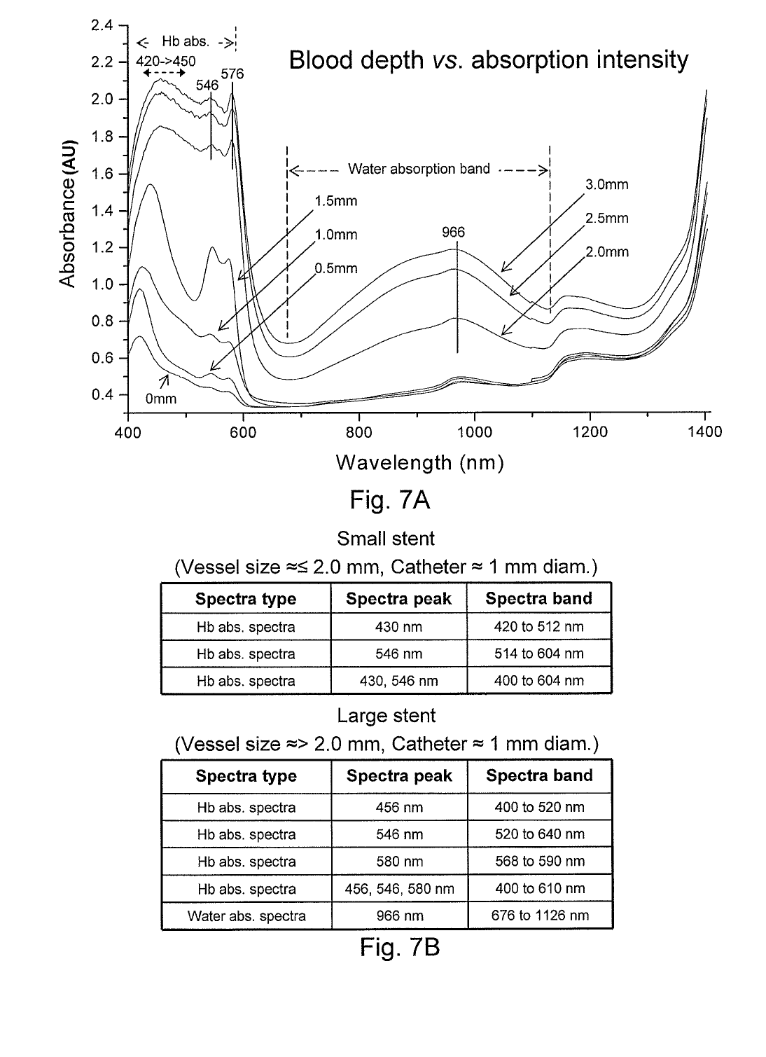

In an embodiment of the invention, source 445 is selected to provide a wavelength range which is substantially absorbed in a blood medium while being highly reflective off of a tissue wall. Such a range can include, for example, wavelengths within a range of between about 200 and 2500 nanometers (from about the ultra-violet through about the near infrared spectrum). In a further embodiment of the invention, a wavelength range of between about 400 and 1400 nanometers is used. Referring to FIG. 7A, diffuse reflection absorbance spectra in a blood medium was measured ex-vivo through various depths of a blood medium above a layer of blood vessel tissue. Absorbance units are represented by −log₁₀(I/I₀) where I is the intensity of the diffuse reflectance signal and I₀ is the intensity of light before it is incident upon the sample. For depths of about 1.5 mm or less, embodiments of the invention analyze absorbance spectra of between about 400 and 600 nanometers (principally associated with hemoglobin). For depths of greater than about 1.5 mm, embodiments of the invention analyze wavelengths of between about 400 and 1400 nanometers (main contribution from both hemoglobin and water).

In an embodiment, the analysis system can be made to discriminate between relevant data such as for determining the geometry of a vessel (e.g. data from targeted blood and tissue) and other data not relevant such as, for example, data relating to the features of a balloon, stent, and/or coatings of a stent. Such features may include, for example, spectral characteristics and/or “shadows” associated with compoents such as a stent, balloon, or guidewire. These features pose a risk of interfering with received radiation, but this risk can be mitigated or eliminated by programming in a data analysis procedure via the spectroscopic analysis system that compensates for such features. Techniques for discriminating data from potentially interfering features are described in, for example, U.S. Pat. No. 6,615,062 by Ryan et al., the entire contents of which are herein incorporated by reference.

Referring to FIG. 7B, relevant spectra peaks in relation to depths of blood media are compared according to estimated vessel diameter ranges and while assuming a catheter diameter of about 1 mm. Assuming a vessel size of about 2 mm or less, the gap between the peripheral edge of the catheter (including optics) and a vessel wall (including across bifurcations) would be approximately 1.5 mm or less. Thus, in an embodiment of the invention, absorption within a range of wavelengths between about 400 and 600 nm (including, for example, peaks at about 430 and 546 nm) can generally be measured for deployment in vessels of less than about 2 mm. The blood component associated with these absorption peaks will generally be that of hemoglobin (Hb).

Assuming a vessel size of greater than about 2 mm, the gap between the peripheral edge of the catheter and a vessel wall (including across bifurcations) would be approximately 0.5 mm or greater. Thus, in another embodiment of the invention, absorption within a range of wavelengths between about 400 and 1400 nm (including, for example, peaks at about 456, 546, 580, and 966 nm) can generally be measured for deployment in vessels of greater than about 2 mm. The additional peak at about 966 nm for larger diameter vessels will be generally associated with that of water (H₂O) absorption. Components, including sources, detectors, and fiber optics are available for measuring backscattered absorption spectra within these ranges from various commercial vendors including, for example, Ocean Optics Inc. of Dunedin, Fla.

While a system such as catheter system 400 would generally be of greater cost and complexity than a simpler system such as catheter system 300 of FIGS. 5A-5B, the dual-fiber arrangement of system 400 can likely provide greater accuracy and detailed information. A multi-fiber system allows for the previously disclosed benefits of improved control over the distribution to collection path and enabling the use of a greater and more dynamic range of wavelength ranges available through absorbance spectra analysis. In addition, data from advanced forms of absorbance and other spectroscopic techniques enabled by a multi-fiber system can provide more extensive information relating to tissue and blood characteristics such as, for example, those referenced Ryan '500, incorporated by reference above.

Referring to FIG. 8A, a combined transmission and collection fiber 563 is shown within the distal end of a balloon catheter 500 in an embodiment of the invention. Referring also to FIG. 8B, a side perspective view of the single-fiber embodiment of the invention shown in FIG. 8A with a stent crimped about the catheter is shown. Referring also to FIG. 8C, an illustrative cross-sectional view is shown of the single fiber embodiment of FIGS. 8A-8B. Fiber 563 is affixed along a catheter body 520 about a portion of which is disposed an unexpanded balloon 540. A sample path 577 of source radiation is shown emanating from fiber 563 in a generally radial direction and sample path 573 of collected radiation is shown directed back to fiber 563. In embodiments of the invention, this and similar single-fiber optical arrangements can be integrated with, for example, the system disclosed in reference to FIGS. 5A and 5B. In an embodiment of the invention, the tip of fiber 563 is of a “side-fire” beveled type in which a reflective coating can be put over the fiber's terminating end, causing radiation to be directed substantially orthogonally (along a radial direction) with respect to the longitudinal axis of the fiber 563 and catheter 500.

Inside of catheter body 520 is a guidewire sheath 525, through which a guidewire 527 can travel and initially direct the positioning of catheter 500 such as in a percutaneous transluminal angioplasty procedure.

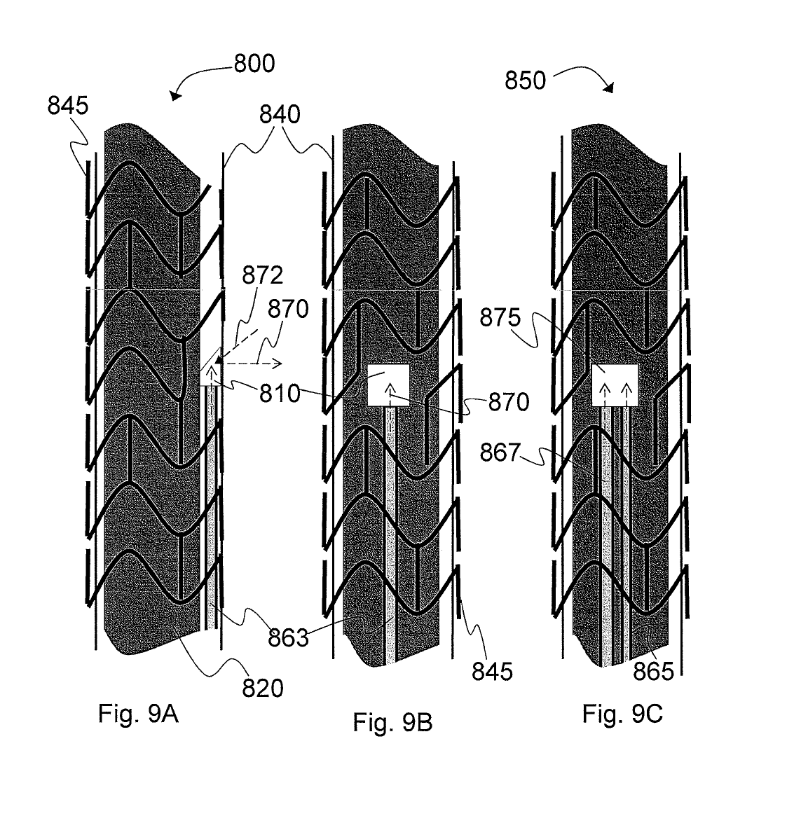

Referring to FIG. 9A, an illustrative side-perspective view is shown of the distal end of a balloon catheter 800 having single transmission/collection fiber 863 terminated with a prism redirector 810 in an embodiment of the invention. Referring also to FIG. 9B, an illustrative head-on perspective view of the embodiment of FIG. 9A is shown. Catheter 800 includes a catheter body 820 about which is disposed a balloon 840 and stent 845 as in an angioplasty catheter. In accordance with single-fiber embodiments of the invention previously disclosed herein, a single fiber 863 is integrated with and runs along the length of catheter 800, terminating at a point in which analysis is to be directed along a generally radial axis with respect to catheter body 820. In order to deliver and collect radiation along a generally radial axis from sheath 820, fiber 863 is terminated with a prism redirector 810. A sample trace 870 of an emission path and a sample trace 872 of a collection path is shown. Numerous micro prisms of appropriate size and material that can be adapted for attachment to optical fibers are commercially available from, for example, Nippon Electric Glass of Shigo, Japan and Tower Optical Corporation of Boynton Beach, Fla.

Referring further to FIG. 9C, an illustrative head-on perspective view of the distal end of a balloon catheter 850 having a transmission fiber 865 and collection fiber 867 terminated with a prism redirector 875 is shown in an embodiment of the invention. This embodiment of the invention can operate in accordance with, for example, various multiple-fiber embodiments of the invention disclosed herein such as described in reference to FIGS. 6A-6B. Adjustments to the prism material, angle, shape, and size can be made to optimize the path of radiation traveling from and to fibers 865 and 867.

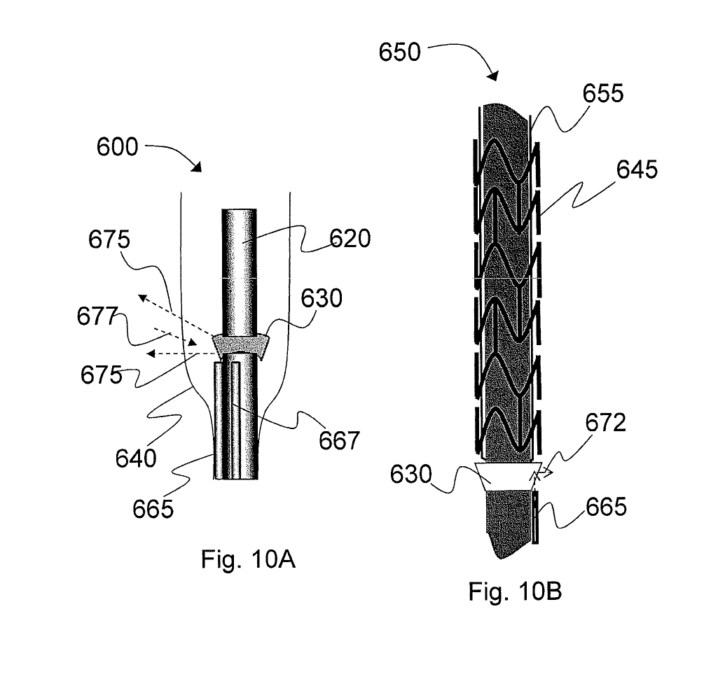

Referring to FIG. 10A, a dual-fiber embodiment of the invention includes a distribution fiber 665 and a collection fiber 667 arranged along a side of a catheter body 620 of a balloon catheter 600 with a balloon 640. A cone-shaped optical redirector 630 located within balloon 640 can direct radiation between fibers 665 and 667 and surrounding blood and tissue. Referring to FIG. 10B, cone-shaped optical redirector 630 can be arranged with the probe ends of the distribution and/or collection fibers 665 and 667 at positions along the catheter that are longitudinally separated from unexpanded balloon 640. For example, the distal end of a catheter 650 includes a cone shaped reflector 630 positioned about the base of a balloon 655 and stent 645 to direct radiation along a predominantly radial trajectory such as sample path 672. An embodiment in accordance with FIG. 10B can be useful during procedures such as described in reference to FIGS. 3B, and 4A-4B. Adjustments to the material, angle, shape, and size of optical redirector 630 can be made to optimize the path of radiation traveling from and to fibers 865 and 867.

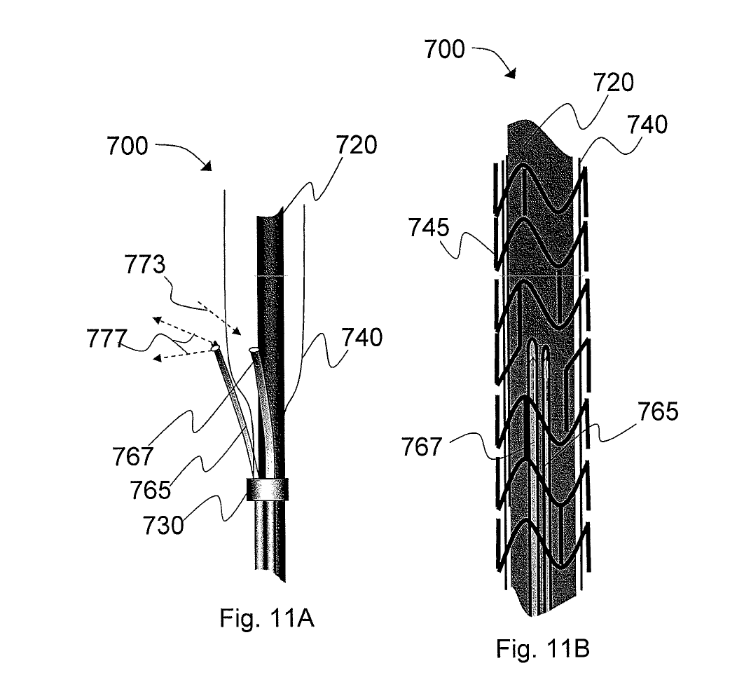

FIG. 11A is an illustrative view of a dual-fiber embodiment of the invention with fibers 765 and 767 arranged along a conduit 720 and terminating on the outside of a balloon 740. FIG. 11B is an illustrative view of the dual-fiber embodiment of FIG. 11A including a crimped stent 745 about balloon 740 and fibers 765 and 767. Sample paths 777 and 773 illustrate radiation being directed from a distribution fiber 765 and being collected by fiber 767 such as in accordance with various embodiments of the invention disclosed herein. The ends of fibers 765 and 767 can be affixed to balloon 740 to help secure them in place during analysis when the balloon is unexpanded. A fiber holder ring 730 secures fibers 765 and 767 along conduit 720 while allowing them to bow when balloon 740 expands.

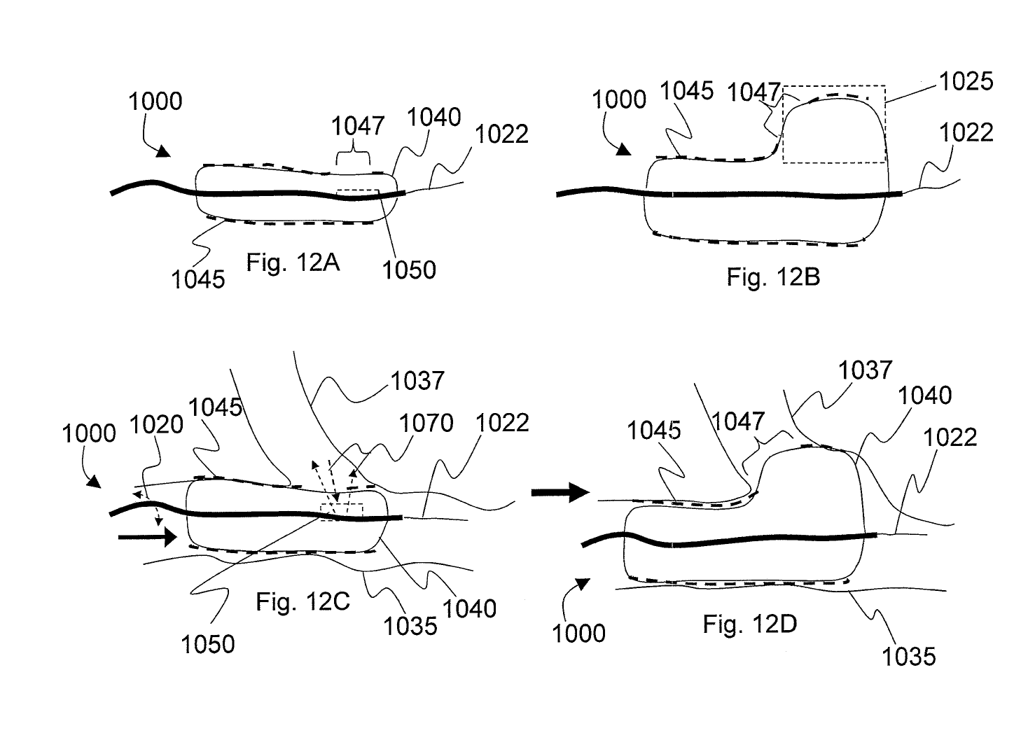

Referring to FIGS. 12A-12D, an embodiment of the invention is shown of a catheter 1000 with a distal end having a pre-shaped balloon 1040 and a stent 1045 with an expanded opening 1047. Catheter 1000 includes a catheter body 1020 and guidewire 1022. FIG. 12A is an illustrated view of catheter 1000 with the pre-shaped balloon 1040 and stent 1045 in an unexpanded state. FIG. 12B is an illustrated view of catheter 1000 of FIG. 12A with the pre-shaped 1040 balloon and stent 1045 in an expanded state (i.e., after expansion with a fluid media such as saline solution). FIGS. 12C-12D are illustrated views of the catheter 1000 of FIGS. 12A-12B deployed in a vessel area 1035 with a branch vessel 1037, with pre-shaped balloon 1040 and stent 1045 in, respectively, unexpanded and expanded states. Balloon 1040 is pre-shaped to form a bulbous area 1025 upon expansion that can widen an opening of an adjacent branch vessel such as branch vessel 1037 and guide a widened opening 1047 of stent 1045 to substantially conform stent with the opening of a branch vessel 1037. A section 1050 of catheter 1000 includes the distal end of a fiber probe arrangement as illustrated in accordance with various embodiments of the present invention disclosed herein for guiding the rotational and/or longitudinal alignment of a catheter.

Referring in particular FIG. 12C, data can be collected from light transmitted and received (such as, for example, along paths 1070) as the distal end of catheter 1000 is rotated about various positions (e.g., as along exemplary rotational path 1020) within a vessel area 1035. The distal ends of one or more fibers can be arranged such that a maximal blood-to-wall span indication will correspond to the rotational and longitudinal position of bulbous area 1025 with the opening of branch vessel 1037 upon balloon 1040's expansion. In an embodiment of the invention, rotational and/or longitudinal movements of catheter 1000 are made in response to such indications in order optimally position bulbuous area 1025. Referring in particular to FIG. 12D, balloon 1040 and stent 1045 are shown expanded within vessel area 1035 while widening the opening of branch vessel 1037. The subsequently widened opening of branch vessel 1037 and expanded opening 1047 of stent 1045 (see FIG. 12D) may be particularly helpful for allowing a subsequent stent (not shown) to be placed therethrough in order to complete a stent bifurcation procedure. In contrast, a typical stent bifurcation procedure will provide a significantly smaller branch vessel opening through which to pass a subsequent stent, thus complicating the procedure and increasing the risks involved.

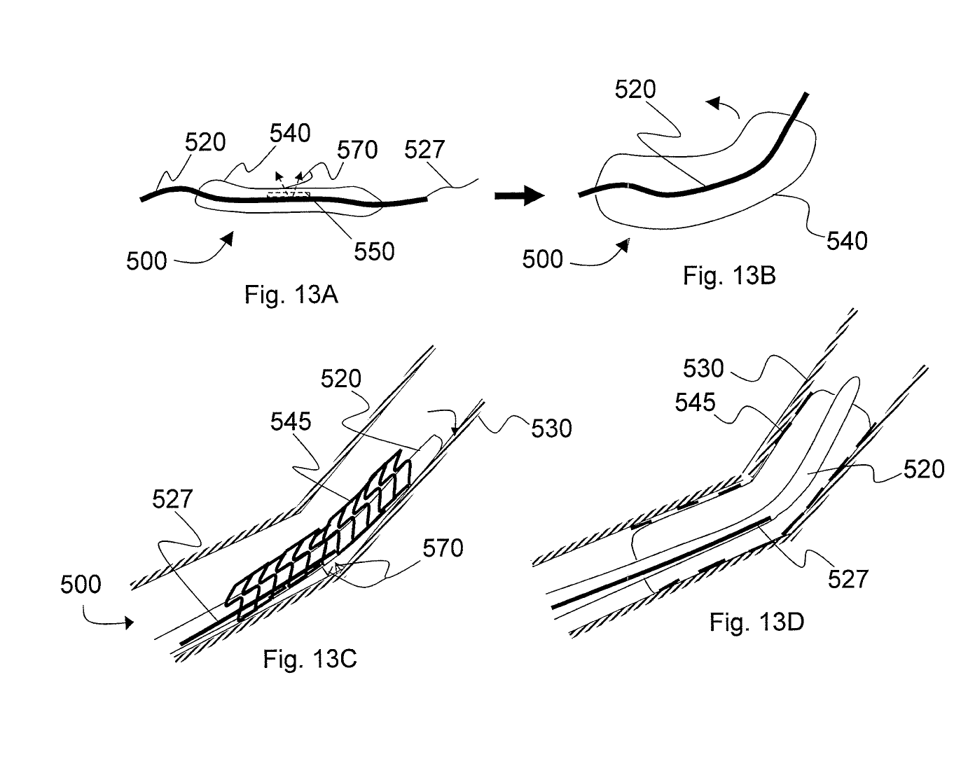

Referring to FIG. 13A and FIG. 13B, the distal end of a balloon catheter 500 with a pre-shaped balloon 540 is shown in, respectively, unexpanded and expanded states according to an embodiment of the invention. Pre-shaped balloon 540 is secured about the distal portion of a catheter body 520. In its unexpanded state (shown in FIG. 13A), pre-shaped balloon 540 remains flexible and compliant so that it may be passed through vessels with a guidewire 527 in a manner, for example, similar to that of typical angioplasty catheters. Catheter 500 includes an optical configuration including a section 550 with the distal end of a fiber probe arrangement such as in accordance with previously described embodiments (e.g., see FIGS. 2, 8-11 and accompanying description) from and to which optical paths 570 are shown directed. When expanded with media (e.g., saline solution), balloon 540 expands to a rigid predetermined shape (shown in FIG. 13B) such as, for example, in accordance with the shape of a highly curved vessel. In an embodiment of the invention, the circumferential position on catheter 500 from which readings of maximum blood depth are taken is aligned with the area of innermost curvature (point of greatest concavity) of balloon 540. A balloon can be pre-shaped (e.g., molded) during manufacture in a manner known to those of skill in the art so as to comply upon expansion with various curvatures such as, for example, increments of varying radii of curvature as needed in a vessel. A catheter with an appropriate balloon shape can be selected based on a preliminary study of the vessel (e.g., an angiogram).

Referring further to FIGS. 13C and 13D, catheter 500 is shown with a crimped stent 545 inserted into a curved vessel area 530. When placed in a significantly curved vessel area during initial positioning, catheter 500 will be pushed toward a side of the vessel wall generally opposite the vessel area's center of curvature. Catheter 500 can then be rotated to various positions within vessel area 530, where readings can be taken in order to determine a position wherein a maximal distance between section 550 and the vessel wall of area 530 is measured. Balloon 540 can then be expanded in place such as shown in FIG. 13D so that the post-expansion shape of balloon 540 substantially conforms with the shape of vessel wall area 530.

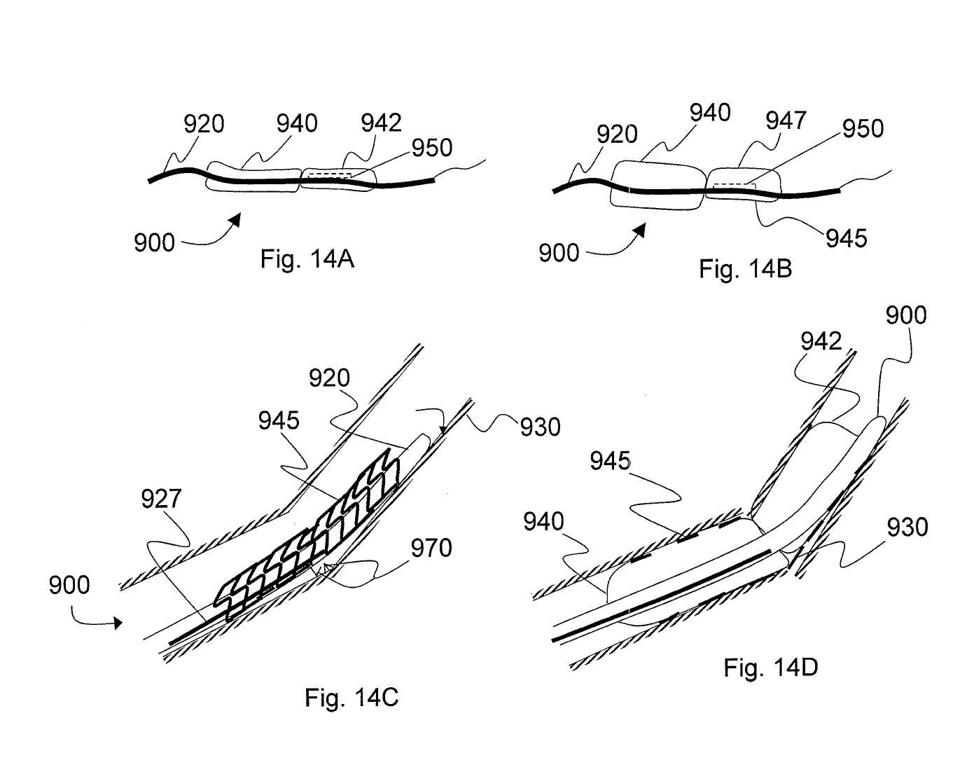

In another embodiment of the invention, FIGS. 14A-14B illustrate views of a balloon catheter 900 having multiple separated balloons 940 and 945 for conformant placement in a curved vessel shown in, respectively, unexpanded and expanded states. FIG. 14C is an illustrative view of the balloon catheter 900 of FIGS. 14A-B, including a crimped stent 945 being rotationally positioned within a curved vessel area 930 according to an embodiment of the invention. FIG. 14D is an illustrative view of the balloon catheter of FIG. 14C in an expanded state within curved vessel area 930. A first balloon 940 is positioned adjacent a second balloon 945, which can straighten and extend in different directions relative to the other, including when balloons 940 and 945 are being expanded. In an embodiment of the invention, balloon 945 is integrated with catheter 900 so that a predominant circumferential portion 947 of balloon 945 is arranged on one side of a catheter body 920. An optimal position of balloon 945 is where the predominant circumferential portion 947 is generally opposite the direction of the bend (opposite the center of curvature) of a target curved vessel area 930 (see, e.g., FIG. 14D). Section 950 of catheter 900 is configured with the probe end of a fiber optic arrangement such as in accordance with embodiments described herein so as to direct radiation 970 to and from the wall of curved vessel area 930. In an embodiment of the invention, measurement of a maximal distance between section 950 and the wall of curved vessel area 930 corresponds to an optimal rotation of balloons 940 and 945 for expansion within curved vessel area 930.

The predicted distribution and collection radiation paths from various embodiments of the catheters disclosed herein can be aligned relative to various features of catheters including therapy delivery components such as, for example, stent strut openings, beveled stent ends, longitudinal stent openings, curvatures of expanded pre-shaped balloons, laser delivery components, tissue extraction components, optical and/or sonic analysis components, and/or other analysis and treatment components.

Embodiments of optical arrangements can incorporate or be combined with other optical arrangements and catheter probe systems such as, for example, those described in previously referenced co-pending application Ryan '500. For example, in an embodiment of the invention, the analysis system provided by Ryan '500 can be combined with embodiments of the present invention in order to perform more detailed and extensive analysis of specific areas circumferentially or longitudinally disposed with respect to the end of a catheter.

It will be understood by those with knowledge in related fields that uses of alternate or varied forms or materials and modifications to the methods disclosed are apparent. This disclosure is intended to cover these and other variations, uses, or other departures from the specific embodiments as come within the art to which the invention pertains.

1. A system for treating a body lumen comprising:
a catheter including a flexible conduit that is elongated along a longitudinal axis and suitable for insertion into a body lumen, the conduit having a proximal end and a distal end;
one or more waveguides integrated with the flexible conduit, the one or more waveguides constructed and arranged to deliver and collect radiation concentrated along a predetermined radial axis of the conduit, the predetermined radial axis of the conduit substantially aligned with respect to at least one analysis or therapy delivery component of the catheter;
at least one radiation source connected to a transmission input of the one or more waveguides integrated with the flexible conduit;
at least one optical detector connected to a transmission output of the one or more waveguides integrated with the flexible conduit.

2. The system of claim 1 further comprising an expandable balloon about the distal end of the conduit, wherein the at least one analysis or therapy delivery component comprises a feature of an angioplasty catheter.

3. The system of claim 2 further comprising an analysis subsystem programmed and configured for determining a relative measure of blood depth outward along the predetermined radial axis from the conduit.

4. The system of claim 2 wherein the feature of said angioplasty catheter comprises a stent.

5. The system of claim 4 wherein the feature of said angioplasty catheter comprises a predetermined opening within said stent.

6. The system of claim 2 wherein the feature of said angioplasty catheter comprises at least a portion of an expandable balloon.

7. The system of claim 6 wherein the feature of said angioplasty catheter comprises a predetermined preformed portion of said expandable balloon.

8. The system of claim 7 wherein said system comprises a controller programmed to process data from said optical detector so as to direct an alignment of said at least one therapy delivery component.

9. The system of claim 1 wherein the radiation source and optical detector are constructed and arranged to induce and detect fluorescence in blood.

10. The system of claim 9 wherein the at least one radiation source is constructed and arranged to supply radiation including a wavelength of about 450 nanometers and wherein the at least one optical detector is constructed and arranged to selectively detect radiation including a wavelength of about 520 nanometers.

11. The system of claim 1 further comprising a spectrometer constructed and arranged to perform spectroscopy on said radiation, said spectroscopy selected from the group of methods including light scatter, optical coherence reflectometry, optical coherence tomography, speckle, correlometry, Raman, and diffuse reflectance spectroscopy.

12. The system of claim 1 further comprising a meter for measuring the level of signal associated with the depth of blood across an area of the body lumen.

13. The system of claim 1 further comprising a controller for adjusting the rotational position of the flexible conduit.

14. The system of claim 1 further comprising a controller for adjusting the longitudinal position of the flexible conduit.

15. The system of claim 1 wherein the at least one analysis or therapy delivery component comprises a predetermined opening of a stent, the predetermined opening formed to substantially conform with an opening of a vessel bifurcation.

16. The system of claim 15 wherein the predetermined opening of the stent comprises a beveled end.

17. The system of claim 1 wherein the at least one analysis or therapy delivery component comprises a portion of an expandable balloon constructed and arranged to bend along the longitudinal axis of the balloon when said balloon is expanded so as to improve conformance of the shape of the expanded balloon with the shape of the lumen.

18. A method of treating a body lumen, the method comprising:
inserting into a body lumen a catheter including a flexible conduit having at least one analysis or therapy delivery component, the flexible conduit comprising one or more waveguides integrated with the flexible conduit, the one or more waveguides constructed and arranged to deliver and collect radiation concentrated along a predetermined radial axis of the conduit, the predetermined radial axis of the conduit substantially aligned with respect to the at least one analysis or therapy delivery component of the catheter, at least one radiation source connected to a transmission input of the one or more waveguides integrated with the flexible conduit, and at least one optical detector connected to a transmission output of the one or more waveguides integrated with the flexible conduit;
maneuvering the conduit into a designated region of the body lumen designated for analysis or treatment;
optimizing a positional alignment of the at least one analysis or therapy delivery component within the body lumen wherein optimizing the positional alignment comprises:
moving the flexible conduit within the body lumen;
measuring and analyzing optical signals collected through the one or more waveguides;
comparing the analysis of the optical signals with the position of flexible conduit; and
repeating the steps of moving the flexible conduit within the body lumen, measuring and analyzing optical signals collected through the one or more waveguides, comparing the analysis of the optical signals with the position of flexible conduit until an optimal alignment of the at least one analysis or therapy delivery component is obtained; and
activating the at least one analysis or therapy delivery component.

19. The method of claim 18 wherein the at least one analysis or therapy delivery component comprises a pre-formed area of an expandable balloon constructed and arranged to dilate an adjacent opening of a vessel bifurcation.

20. The method of claim 18 wherein measuring and analyzing optical signals collected through the one or more waveguides comprises delivering radiation of one or more wavelengths so as to induce fluorescence, and measuring the intensity of radiation generated from the fluorescence.

21. The method of claim 20 wherein the one or more wavelengths of radiation generated to induce fluorescence includes a wavelength of 450 nanometers and wherein the at least one wavelength of radiation generated from the fluorescence includes a wavelength of 520 nanometers.

22. The method of claim 18 wherein optimizing the positional alignment comprises optimizing rotational alignment of the flexible conduit.

23. The method of claim 18 wherein optimizing the positional alignment comprises optimizing longitudinal alignment of the flexible conduit.
